(19) United States
(12) Patent Application Publication
(10) Pub. No.: US 2023/0393128 A1
(43) Pub. Date: Dec. 7, 2023

(54) POLYMERIC MATERIAL-COATED NANOPARTICLE COMPLEX FOR ISOLATION OF BIOLOGICAL TARGET AND PREPARATION METHOD THEREFOR

(71) Applicants: RESEARCH & BUSINESS FOUNDATION SUNGKYUNKWAN UNIVERSITY, Suwon-si (KR); KINGOBIO INC., Seoul (KR)

(72) Inventors: Sungsu PARK, Seoul (KR); Jinyeop LEE, Suwon-si (KR); Tepeng WU, Seoul (KR); Byungjoon NA, Suwon-si (KR)

(73) Assignees: RESEARCH & BUSINESS FOUNDATION SUNGKYUNKWAN UNIVERSITY, Suwon-si (KR); KINGOBIO INC., Seoul (KR)

(21) Appl. No.: 18/451,734

(22) Filed: Aug. 17, 2023

Related U.S. Application Data
(63) Continuation of application No. PCT/KR2022/002367, filed on Feb. 17, 2022.

(30) Foreign Application Priority Data
Feb. 17, 2021 (KR) 10-2021-0021476
Feb. 16, 2022 (KR) 10-2022-0020257

(51) Int. Cl. G01N 33/543; G01N 33/548; G01N 33/551

(52) U.S. Cl. CPC G01N 33/54346; G01N 33/548; G01N 33/551

(57) ABSTRACT

The present invention relates to a nanoparticle complex for isolation of a biological target and a preparation method therefor and, more specifically, to a nanoparticle complex in which a receptor is conjugated to a polymer-coated nanoparticle, a preparation method therefor, and a use thereof.

### CROSS-REFERENCE TO RELATED APPLICATIONS

The present application claims the benefit of priority to Korean Patent Application Nos. 10-2021-0021476 and 10-2022-0020257 filed in the Korean Intellectual Property Office on Feb. 17, 2021 and Feb. 16, 2022, respectively, and all the contents disclosed in the specifications and drawings of those applications are incorporated in this application.

### TECHNICAL FIELD

The present invention relates to a nanoparticle complex for isolation of a biological target, a preparation method therefor, and the like, and more specifically, to a nanoparticle complex with a receptor bonded to polymer-coated nanoparticles, in which the dispersed state of the nanoparticle complex can be effectively maintained by reducing the matrix effect in a biological sample in which various components are present in combination, thereby isolating a biological target with high efficiency.

### BACKGROUND ART

As interest in health and diseases is increasing at the present time, research has been actively conducted on methods capable of managing health and reducing treatment costs through early diagnosis of diseases. Among them, there have been many attempts to detect diseases by a method of recognizing or isolating specific biomarkers from biological samples in which various components are present in combination using nanoparticles, but due to the presence of large amounts of other impurities including cellular materials in the form of proteins, nucleic acids, and carbohydrates in biological samples such as blood, saliva, and urine, the matrix effect of nanoparticles can cause particles to aggregate, so that not only is the detection sensitivity of a target material remarkably reduced, but also it is difficult to isolate the target material efficiently (Korean Patent No. 10-1646610).

Therefore, if particles can be efficiently dispersed by preventing the matrix effect to suppress the aggregation of the particles in a biological sample, it is expected that not only can the binding force to a target be increased to remarkably increase detection sensitivity, but also the target material can be isolated and/or detected with high efficiency.

### Technical Problem

The present invention has been devised to solve the problems in the related art as described above, and relates to a nanoparticle complex having a receptor coupled to a nanoparticle coated with a polymer, and has an object to provide a nanoparticle complex in which the dispersion force of the nanoparticles is enhanced by coating the nanoparticles to suppress the matrix effect, thereby remarkably increasing detection sensitivity, a preparation method therefor, a use thereof, and the like.

However, the technical problems which the present invention intends to solve are not limited to the technical problems which have been mentioned above, and other technical problems which have not been mentioned will clearly be understood by those with ordinary skill in the art to which the present invention pertains from the following description.

### Technical Solution

The present invention provides a nanoparticle complex for detecting or isolating a target material from a biological sample. The nanoparticle complex can prevent the matrix effect in a biological sample by preferably coating the surface of nanoparticles with a polymer material (macromolecule), and a receptor for detecting or isolating a target material can be bound to the macromolecule to specifically bind to the target material. Also, the polymer material may be preferably any one or more selected from the group consisting of polydopamine, polyethylene glycol, polyetherimide, polyvinyl alcohol, casein, dextran, and chitosan, and is more preferably polydopamine or polyethylene glycol, but is not limited thereto as long as it is a polymer material that can coat nanoparticles by a self-polymerization reaction or can bind to nanoparticles having a thiol group. In addition, the coating may have preferably a thickness of 1 to 500 nm, more preferably 1 to 100 nm, and even more preferably 1 to 30 nm, but the thickness is not limited thereto as long as the coating can prevent the matrix effect.

In an exemplary embodiment of the present invention, the coating may be coated by mixing a monosaccharide for binding to lectin, and the like with a polymer material, and the monosaccharide may include mannose, glucose, fructose, glucosamine, and the like, but is not limited thereto as long as the coating is a monosaccharide capable of binding to lectin by electrostatic binding.

In another exemplary embodiment of the present invention, the bond may be a bond in the form of any one or more selected from the group consisting of ionic bonds, covalent bonds, coordinate bonds, hydrogen bonds, hydrophobic interactions, van der Waals interactions, and bonds by a linker, but is not limited thereto as long as it is in a form known as a method for binding a polymer material to a receptor.

In still another exemplary embodiment of the present invention, the bond by a linker is characterized by a bond by a 1-ethyl-3-(3-dimethylaminopropyl)carbodiimide (EDC)/N-hydroxysuccinimide (NHS) linker or a thiol group.

In yet another exemplary embodiment of the present invention, the receptor may be preferably an antibiotic, an antibody, a nucleic acid, a protein, lectin, and the like, which specifically bind to a target material, and the nucleic acid may be plasmid DNA, cDNA, siRNA, miRNA, antisense RNA, an aptamer, an oligonucleotide, and the like. However, the receptor is not limited thereto as long as it can specifically bind to a target material to detect or isolate the target material.

In yet another exemplary embodiment of the present invention, the target material may be a pathogen, a virus, a cell, a protein, a nucleic acid, an antigen, a glycoprotein, a metal, and the like, and is not limited thereto as long as it is a biomarker that can be used to diagnose diseases.

In yet another exemplary embodiment of the present invention, the nanoparticles may be silica nanoparticles, iron oxide nanoparticles, polystyrene nanoparticles, and the like, but are not limited thereto as long as they can be coated with a polymer material.

In yet another exemplary embodiment of the present invention, the diameter of the nanoparticle complex may be preferably 200 to 1,000 nm, more preferably 200 to 800 nm, but is not limited thereto.

In yet another exemplary embodiment of the present invention, the biological sample may be preferably blood, saliva, bone marrow fluid, lymphatic fluid, urine, amniotic fluid, mucosal fluid, peritoneal fluid, and the like, and is not limited thereto as long as it is a complex mixture of various materials such as cells, nucleic acids, proteins, extracellular vesicles and impurities.

Further, the present invention provides a method for preparing a nanoparticle complex, the method including the following steps: a) preparing polydopamine-coated nanoparticles by adding and dispersing nanoparticles and an acid-base catalyst in a polydopamine solution; and b) adding a receptor to the polydopamine-coated nanoparticles and reacting the resulting mixture to prepare a receptor-bound polydopamine-coated nanoparticle complex.

In an exemplary embodiment of the present invention, the nanoparticles and polydopamine in Step a) may be mixed at a weight ratio of 1:0.01 to 1:1.5, and a monosaccharide may be further added to the polydopamine solution in Step a) at a weight ratio of 1:0.5 to 1:1.5.

In another exemplary embodiment of the present invention, the nanoparticles may be preferably iron oxide nanoparticles, polystyrene nanoparticles, and the like, but are not limited thereto as long as they can be coated by a self-polymerization reaction with a polymer material.

In still another exemplary embodiment of the present invention, in Step a), nanoparticles and an acid-base catalyst may be dispersed in a polydopamine solution for 4 hours to 36 hours, and in Step b) the resulting mixture may react for 1 hour to 5 hours.

In addition, the present invention provides a method for preparing a nanoparticle complex, the method including the following steps: a) preparing polymer material-coated nanoparticles by adding a polymer material to silica shell nanoparticles having a thiol group and dispersing the same; and b) adding a receptor to the coated nanoparticles and reacting the resulting mixture to prepare a receptor-bound polymer material-coated nanoparticle complex, in which the polymer material may be polyethylene glycol, polyetherimide, polyvinyl alcohol, casein, dextran, chitosan, and the like.

In an exemplary embodiment of the present invention, in Step b), 1-ethyl-3-(3-dimethylaminopropyl)carbodiimide (EDC) and N-hydroxysuccinimide (NHS) may be added along with a receptor and the resulting mixture may be reacted.

In another exemplary embodiment of the present invention, the nanoparticles and polyethylene glycol or polyvinyl alcohol in Step a) may be mixed at a weight ratio of 1:1 to 1:5 and dispersed for 4 to 36 hours.

In still another exemplary embodiment of the present invention, Step b) may be performed for 1 hour to 5 hours.

In addition, the present invention provides a method for detecting or isolating a target material from a biological sample using the nanoparticle complex. The method is characterized by including a step of treating and reacting a biological sample in vitro with the nanoparticle complex.

Furthermore, the present invention provides a method for diagnosing an infectious disease or a method for providing information on diagnosing an infectious disease, using the nanoparticle complex. The method is characterized by including a step of treating and reacting a biological sample in vitro with the nanoparticle complex.

Further, the present invention provides a use of the nanoparticle complex for detecting or isolating a target material from a biological sample.

In addition, the present invention provides a use of the nanoparticle complex for diagnosing an infectious disease.

Furthermore, the present invention provides a kit for detecting or isolating a target material from a biological sample, including the nanoparticle complex as an active ingredient.

Further, the present invention provides a kit for diagnosing an infectious disease, including the nanoparticle complex as an active ingredient.

### Advantageous Effects

The nanoparticle complex according to the present invention can be easily prepared by effectively binding various receptors to the nanoparticles using polymer-coated nanoparticles, and through this, the nanoparticle complex according to the present invention can be effectively used for diagnosing various diseases using biomarkers, such as cancer, as well as infectious diseases caused by pathogenic bacteria, viruses, and the like. In addition, by preventing the matrix effect which occurs in a biological sample using nanoparticles coated with a polymer, the aggregation among particles does not occur even though the biological sample is directly treated with the nanoparticle complex without any pre-treatment of the biological sample, and through this, the fouling phenomenon can be prevented, thereby remarkably increasing detection sensitivity. Therefore, the nanoparticle complex of the present invention is expected to be widely used for isolation, detection, diagnosis, treatment, and the like of various biological targets using a receptor.

### BRIEF DESCRIPTION OF DRAWINGS

FIG. 1 is a set of views illustrating the results of observing nanoparticles coated with a polymer material according to an embodiment of the present invention using a scanning electron microscope.

FIG. 2 is a set of views illustrating the results of observing nanoparticles coated with a polymer material according to an embodiment of the present invention using a transmission electron microscope.

FIG. 3 is a view schematically illustrating a method for preparing a PDA-coated nanoparticle complex bound to an antibody or an antibiotic and a mechanism of binding to a target according to an embodiment of the present invention.

FIG. 4 is a view schematically illustrating a method for preparing a Man/PDA-coated nanoparticle complex bound to lectin and a mechanism of binding to a target according to an embodiment of the present invention.

FIG. 5 is a view schematically illustrating a method for preparing a PEG-coated nanoparticle complex bound to an antibody or an antibiotic according to an embodiment of the present invention.

FIG. 6 is a set of views illustrating the results of confirming the dispersibility of nanoparticles according to an embodiment of the present invention.

FIG. 7 is a view illustrating the results of confirming the ability to isolate a target using a polymer material-coated nanoparticle complex coated bound to an antibiotic according to an embodiment of the present invention.

### DETAILED DESCRIPTION

The nanoparticle complex of the present invention is a nanoparticle complex in which a receptor is bound to a polymer-coated nanoparticle, and may remarkably improve the detection sensitivity of a target material included in a biological sample by coating the surface of the nanoparticles with a polymer such as PDA, PEG, and PVA to a nanometer-sized thickness to prevent the matrix effect in the biological sample, so that the nanoparticle complex of the present invention can be effectively used for diagnosing various diseases because it can detect and/or isolate the target material in a small amount with high accuracy.

As used herein, the “specifically binding” refers to specifically interacting and binding to a target material to be isolated or detected, and refers to a form in which a receptor bound to the nanoparticle complex of the present invention and the target material are bound. The target material may include a material or a biomarker, and the like, which are generally used to diagnose a disease, such as a nucleic acid, an antigen, a protein, cells, cancer cells, a pathogen, a virus, and a metal included in a biological sample.

As used herein, the “receptor” is a generic term for a material that specifically binds to the target material, and is preferably an antibiotic, an antibody, a nucleic acid, a protein, lectin, and the like, but is not limited thereto as long as it specifically binds to the target material and can detect or isolate the target material.

As used herein, the “nanoparticles” include all commonly used nano-sized particles, and may be preferably selected from the group consisting of silicon particles, polystyrene particles, latex particles, metal particles, glass particles, magnetic particles, silica shell nanoparticles, and combinations thereof, but are not limited thereto.

As used herein, the “biological sample” can be any biological sample in which the target cells may be present, and may be selected from the group consisting of, for example, a biopsy sample, a tissue sample, a cell suspension in which isolated cells are suspended in a liquid medium, a cell culture, a body fluid, and a combination thereof, and the body fluid may include blood, saliva, bone marrow fluid, lymphatic fluid, urine, amniotic fluid, mucosal fluid, peritoneal fluid, and the like.

As used herein, the “coating” refers to applying a thin film to the surface of nanoparticles using a polymer material, and the thin film may have a thickness of 1 to 500 nm, 1 to 200 nm, 1 to 100 nm, 1 to 50 nm, or 1 to 30 nm, but the thickness is not limited thereto.

As used herein, the “kit” refers to a device for detecting or isolating a target material from a biological sample, including the nanoparticle complex of the present invention as an active ingredient, and is not limited as long as it is in a form that enables confirmation of the presence or absence of a target material or the amount of a target material from a biological sample isolated from an organism. The kit may be used in various diagnostic fields by confirming the presence or absence of the target material bound to the nanoparticle complex of the present invention or the amount of the target material, and preferably, it may also be used to diagnose an infectious disease.

As used herein, the “infectious disease” refers to any disease resulting from infection with pathogenic microorganisms, and the pathogenic microorganisms may be bacteria, viruses, protozoa, fungi, and the like, but are not limited thereto as long as they are microorganisms known to be capable of infecting living organisms and causing infectious diseases.

Hereinafter, preferred examples for helping with understanding of the present invention will be suggested. However, the following examples are provided only so that the present invention may be more easily understood, and the content of the present invention is not limited by the following examples.

### EXAMPLES

### Example 1: Preparation of Nanoparticles Coated with Polymer Material

Polymer material-coated nanoparticles were prepared to prepare nanoparticles for preventing the matrix effect, which is a problem when analyzing a biological sample in which various components are mixed in a complex manner. To select a polymer material that could most effectively prevent the matrix effect, nanoparticles coated with a polymer material were prepared using polydopamine (PDA), polyethylene glycol (PEG), polyetherimide (PEI), polyvinyl alcohol (PVA), casein, dextran, and chitosan.

Iron oxide nanoparticles were prepared by a commonly known method. More specifically, 100 mg of Fe₃O₄ powder (100 nm) was added to 40 mL of a 1 M hydrochloric acid (HCl) solution, and the resulting mixture was mixed, and then reacted at room temperature for 1 hour. After the particles were captured through a permanent magnet, the remaining solution was removed, and the captured particles were added to 40 mL of phosphate buffered saline (PBS, pH 7.4) and vortexed for 1 minute, and washed three times by a method of collecting only particles again using the permanent magnet. Finally, the prepared iron oxide nanoparticles were placed in 10 mL of phosphate buffered saline and stored.

In order to prepare PDA-coated nanoparticles, first, 50 mg of dopamine was added to a buffer solution to which 25 mL of Tris-HCl buffer (pH 8.5) or acetic acid was added as an acid-base catalyst to prepare a PDA solution, and thereafter, nanoparticles such as iron oxide nanoparticles or polystyrene nanoparticles were added and dispersed by ultrasonic treatment for 16 hours to prepare PDA-coated nanoparticles (NPs@PDA) through a self-polymerization reaction. After the particles were captured through a permanent magnet, the remaining solution was removed, and the captured particles were added to 40 mL of phosphate buffered saline (PBS, pH 7.4) and vortexed for 1 minute, and washed three times by a method of collecting only particles again using the permanent magnet. Finally, the prepared nanoparticles coated with PDA were placed in 1 mL of phosphate buffered saline and stored. In this case, PDA-coated nanoparticles were prepared by varying the content ratio of nanoparticles and PDA, and it was confirmed that nanoparticles coated with PDA were effectively prepared when the content ratio of the nanoparticles and PDA was 1:0.01 to 1:1.5, and the nanoparticles aggregated when the weight ratio exceeded 1:1.5, and the PDA coating was not sufficient when the weight ratio was less than 1:0.01. It was confirmed that the coating thickness of PDA was decreased by decreasing the self-polymerization reaction time, and that the coating thickness of PDA was also decreased when the content ratio of PDA was decreased. As a result, it was confirmed that the time required for coating with a thickness of 1 to 30 nm was 4 to 36 hours.

Silica shell nanoparticles having a thiol group (NPs@SiO₂) were used to coat the nanoparticles with a polymer material that binds to the thiol group, and polyethylene glycol, polyetherimide, polyvinyl alcohol, casein, dextran, and chitosan were used as the polymer material. For the silica shell nanoparticles, 0.5 mL of a NH₄OH solution to which tetra ethyl ortho silicate (TEOS) was added was added to 80 mg of magnetic nanoparticles (MNPs) such as iron oxide or polystyrene nanoparticles, and the resulting mixture was reacted for 16 hours to prepare nanoparticles in which a silica shell was formed. 40 mL of mercaptoethanol (MA, 80% v/v), 400 mg of 1-ethyl-3-(3-dimethylaminopropyl)carbodiimide (EDC), 40 mL of dimethylformamide (DMF) and 80 mg of 4-dimethylaminopyridine (DMAP) were added to the prepared silica-shell nanoparticles and the resulting mixture was reacted for 16 hours to form thiol groups on the silica shell surface. Thereafter, 2 mg of silica shell nanoparticles with thiol groups on the surface, 0.6 mg of PEG powder (molecular weight: 6,000), PEI powder, dextran, casein, or chitosan were added to 1 mL of phosphate buffered saline (pH 7.4) and mixed, and then the resulting mixture was reacted for 16 hours to prepare nanoparticles coated with a polymer material. In this case, polymer material-coated nanoparticles were prepared by varying the content ratio of nanoparticles and the polymer material, and it was confirmed that nanoparticles coated with the polymer material are effectively prepared when the content ratio of the nanoparticles and the polymer material was 1:1 to 1:5, and the nanoparticles aggregated when the weight ratio exceeded 1:5, and the polymer material coating was not sufficient when the weight ratio was less than 1:1. Also, it was confirmed that the coating thickness of the polymer material was reduced by reducing the reaction time to 12 hours, 8 hours, and 4 hours, and the coating thickness of the polymer material was also reduced when the content ratio of the polymer material was reduced.

Then, 2 mg of 1-ethyl-3-(3-dimethylaminopropyl) carbodiimide (EDC) and 2 mg of N-hydroxysuccinimide (NHS) were added to the nanoparticles coated with the polymer material and the resulting mixture was reacted at room temperature for 6 hours to bind to a linker for amine coupling, such that the receptor could bind. After the particles were captured through a permanent magnet, the remaining solution was removed, and the captured particles were added to 40 mL of phosphate buffered saline (pH 7.4) and vortexed for 1 minute, and washed three times by a method of collecting only particles again using the permanent magnet. Finally, the prepared nanoparticles coated with the polymer material were placed in 1 mL of phosphate buffered saline and stored.

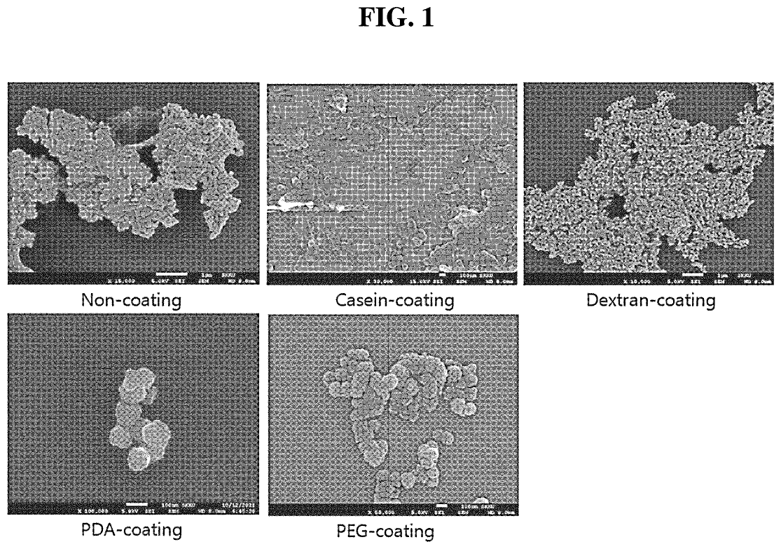

Hereinafter, in all experiments, uncoated silica nanoparticles were used as a control. Nanoparticles coated with each polymer material prepared by the above methods were observed using a scanning electron microscope (SEM). The results are illustrated in FIG. 1.

As shown in FIG. 1, it could be confirmed that the uncoated nanoparticles and the nanoparticles coated with the polymer material have different surface textures, thereby confirming that the nanoparticles were coated with the polymer material. It was also confirmed that nanoparticles coated with casein or dextran among polymer material-coated nanoparticles aggregated together, and particularly, that the aggregation phenomenon was the greatest in the case of casein. Conversely, it was confirmed that PDA, PEG, PEI, and chitosan hardly caused aggregation.

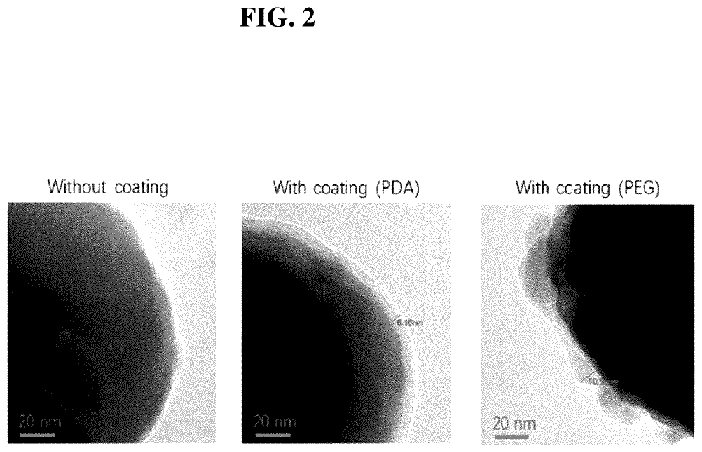

In addition, nanoparticles coated with PDA or PEG were observed using a transmission electron microscope (TEM) in order to observe the nanoparticles coated with a polymer material in more detail. The results are illustrated in FIG. 2.

As illustrated in FIG. 2, it was confirmed that a thin film was formed on the surface of the PDA- or PEG-coated nanoparticles, while no film was formed on the control silica nanoparticles. Through the above results, nanoparticles were coated with PDA or PEG to form a film, and through this, it was confirmed that PDA-coated nanoparticles (NPs@PDA) and PEG-coated nanoparticles (MNPs@SiO2-PEG) were successfully prepared.

### Example 2: Preparation of Nanoparticle Complex

In order to isolate, purify and detect a target from a biological sample, a nanoparticle complex, in which a receptor capable of binding to pathogens, cells, biomarkers (nucleic acids, enzymes, proteins, and the like) was bound to nanoparticles coated with a polymer material prepared in the same manner as in Example 1, was prepared.

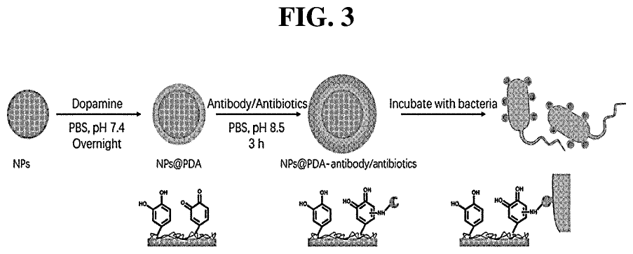

After antibodies or antibiotics were added to the PDA-coated nanoparticles, the resulting mixture was dispersed at 4° C., and reacted for 2 hours or more to prepare a PDA-coated nanoparticle complex in which the antibodies or antibiotics were bound to PDA. The method for preparing a PDA-coated nanoparticle complex bound to an antibody or antibiotic is schematically illustrated in FIG. 3.

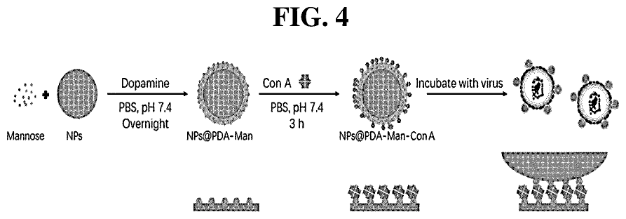

To bind lectin targeting viruses, mannose (Man), which is a type of monosaccharide, was mixed with PDA at a weight ratio of 1:1, and then iron oxide nanoparticles were added thereto and the resulting mixture was dispersed by ultrasonic treatment for 16 hours, nanoparticles coated with Man and PDA (NPs@PDA-Man) were prepared through a self-polymerization reaction, Concanavalin A (ConA) was added to the prepared coated nanoparticles, and the resulting mixture was reacted for 3 hours to prepare a Man- and PDA-coated nanoparticle complex covalently bound to lectin. The method for preparing a Man/PDA-coated nanoparticle complex bound to lectin is schematically illustrated in FIG. 4.

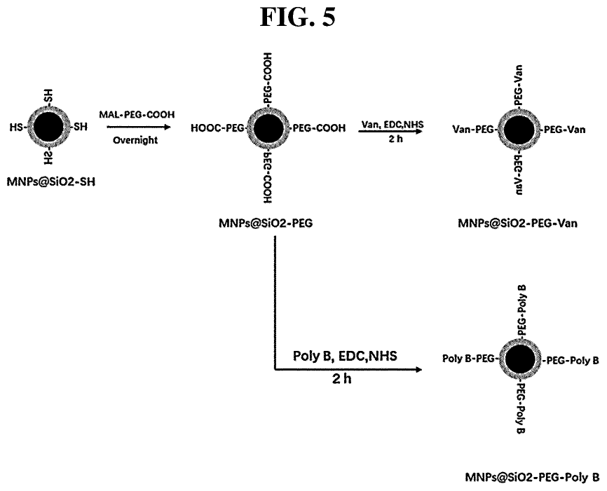

In order to bind antibiotics or antibodies to PEG-coated nanoparticles bound to EDC/NHS linkers, the nanoparticles were treated with an antibiotic such as vancomycin and polymyxin B and reacted at 4° C. for 2 hours to prepare a nanoparticle complex in which an antibiotic is bound to PEG. The method for preparing a PEG-coated nanoparticle complex bound to an antibody or antibiotic is schematically illustrated in FIG. 5.

The prepared nanoparticle complexes were each stored in phosphate buffered saline until use.

### Example 3: Characterization of Nanoparticle Complex

3.1. Confirmation of Dispersibility of Nanoparticles

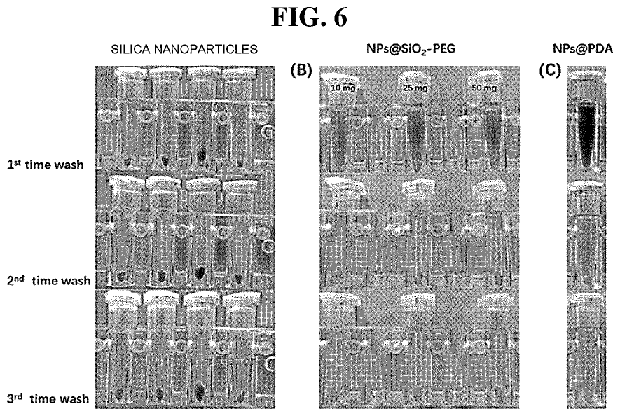

In order to confirm the dispersibility of the nanoparticles prepared in the same manner as in Example 1 in a biological sample, PEG-coated silica nanoparticles (NPs@SiO₂—PEG), which were confirmed to have the least aggregation phenomenon, and PDA-coated nanoparticles (NPs@PDA) were each added to whole blood, and the degree of aggregation between particles according to the number of washes was confirmed. More specifically, each type of nanoparticle was added to 1 mL of phosphate buffered saline and vortexed for 15 seconds, and then the nanoparticles were captured using a magnetic rack, and then the supernatant was removed, and the process of adding phosphate buffered saline was repeated three times. Uncoated nanoparticles were used as a control. The results are illustrated in FIG. 6.

As illustrated in FIG. 6, it was confirmed that uncoated silica nanoparticles aggregated and sank to the bottom, whereas PEG- or PDA-coated nanoparticles were dispersed in the blood and did not aggregate. From the above results, it could be confirmed that the nanoparticles coated with the polymer material of the present invention do not aggregate together nor exhibit the matrix effect in the biological sample, similar to the results observed by SEM.

3.2. Confirmation of Target Isolation Ability

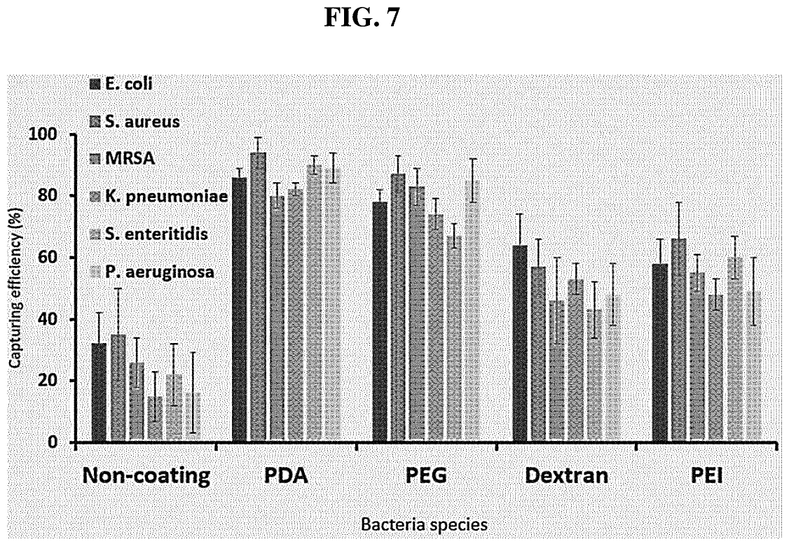

In order to confirm the target isolation ability of the nanoparticle complex prepared in the same manner as in Example 2, the isolation ability of bacteria contained in whole blood was confirmed. More specifically, whole blood was mixed with each pathogenic bacterium or virus, treated with nanoparticle complexes, and reacted for 20 minutes, and then the nanoparticle complex was isolated. The amount of pathogens bound to the isolated nanoparticle complex was confirmed by performing PCR using bacterial universal 16s rDNA primers, and the amount of pathogens bound to nanoparticles is expressed in % based on the amount of pathogens in samples to which nanoparticles were not added. As a target strain, Escherichia coli O157:H7 (E. coli), Staphylococcus aureus (S. aureus), Methicillin-resistant Staphylococcus aureus (MRSA), which is an antibiotic-resistant strain, Klebsiella pneumonia (K. pneumonia), Pseudomonas aeruginosa (P. aeruginosa) and Salmonella enteritidis (S. Enteritidis) were used, and as nanoparticles, a nanoparticle complex in which receptors are bound to uncoated silica nanoparticles, a nanoparticle complex in which receptors are bound to PDA-coated nanoparticles, a nanoparticle complex in which receptors are bound to PEG-coated nanoparticles with EDC/NHS linkers, a nanoparticle complex in which receptors are bound to dextran-coated nanoparticles, and a nanoparticle complex in which receptors are bound to PEI-coated nanoparticles were used. As the receptor, vancomycin or polymyxins B was used. The results are illustrated in FIG. 7.

As illustrated in FIG. 7, it was confirmed that the nanoparticles not coated with a polymer material exhibited a capture efficiency of 40% or less, whereas the nanoparticles coated with a polymer material exhibited a capture efficiency of 40% or more. Among them, it was confirmed that PDA- or PEG-coated nanoparticles exhibited a capture efficiency of 80% or more within 20 minutes, thereby exhibiting the highest capture efficiency.

In addition, in order to confirm the isolation ability using antibodies, influenza A virus subtype H1N1, an influenza virus, was mixed in saliva and treated with a PDA-coated nanoparticle complex bound to an anti-H1N1 antibody (Santa Cruz Biotechnology), and reacted for 20 minutes, and then the nanoparticle complex was isolated. Then, the amount of virus bound to the isolated nanoparticle complex was confirmed using an antibody. As a result, it was confirmed that when a virus was isolated using a PDA-coated nanoparticle complex bound to an anti-H1N1 antibody, approximately 90% of the virus was isolated within 20 minutes, whereas when an uncoated nanoparticle complex bound to an anti-H1N1 antibody was used, approximately 40% of the virus was isolated within 20 minutes, and thus, capture efficiency was remarkably increased in the case of nanoparticles coated with a polymer material.

Through the above results, it could be confirmed that the nanoparticles coated with the polymer of the present invention may be present in an efficiently dispersed state by not only suppressing the aggregation phenomenon between nanoparticles in an aqueous solution, but also preventing the matrix effect in biological samples in which various components such as blood, saliva, and urine are present in a complex manner to suppress aggregation between particles, and thus, the isolation and purification efficiency of a target in an actual biological sample can be remarkably increased, and through this, the detection sensitivity, detection accuracy, diagnostic accuracy, and the like of analysis technology can be remarkably increased ultimately. Furthermore, it could be confirmed that the nanoparticles coated with the polymer of the present invention can be effectively applied to various fields to detect various target materials because various types of receptors can be easily bound to the surface of the coated polymer material without any limitation by various known methods.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

Since the nanoparticle complex according to the present invention not only allows various receptors to be easily bound to nanoparticles, but also improves detection sensitivity, it can be effectively used for diagnosing various diseases using biomarkers, such as cancer, as well as infectious diseases caused by pathogens, viruses, and the like. In addition, by changing the receptor type, the nanoparticle complex according to the present invention can be widely used for isolation, detection, diagnosis, treatment, and the like of various biological targets.

1. A nanoparticle complex for detecting or isolating a target material from a biological sample,
wherein the nanoparticle complex is a complex in which the surface of nanoparticles is coated with a polymer material, and comprises a receptor for detecting or isolating a target material bound to the polymer material, and
the polymer material is any one or more selected from the group consisting of polydopamine, polyethylene glycol, polyetherimide, polyvinyl alcohol, casein, dextran, and chitosan.

2. The nanoparticle complex of claim 1, wherein the coating is a mixture of a monosaccharide for lectin binding and a polymer material.

3. The nanoparticle complex of claim 2, wherein the monosaccharide is any one or more selected from the group consisting of mannose, glucose, fructose, and glucosamine.

4. The nanoparticle complex of claim 1, wherein the bond is in the form of any one or more selected from the group consisting of ionic bonds, covalent bonds, coordinate bonds, hydrogen bonds, hydrophobic interactions, van der Waals interactions, and bonds by a linker.

5. The nanoparticle complex of claim 4, wherein the bond by a linker is a bond by 1-ethyl-3-(3-dimethylaminopropyl)carbodiimide (EDC)/N-hydroxysuccinimide (NHS) linker or a thiol group.

6. The nanoparticle complex of claim 1, wherein the receptor is any one or more selected from the group consisting of an antibiotic, an antibody, a nucleic acid, lectin, and a protein, which specifically bind to a target material.

7. The nanoparticle complex of claim 1, wherein the target material is any one or more selected from the group consisting of a pathogen, a virus, a cell, a protein, a nucleic acid, an antigen, and a glycoprotein.

8. The nanoparticle complex of claim 1, wherein the nanoparticles are any one or more type of nanoparticle selected from the group consisting of silica nanoparticles, iron oxide nanoparticles, and polystyrene nanoparticles.

9. The nanoparticle complex of claim 1, wherein the nanoparticle complex has a diameter of 200 to 1,000 nm.

10. The nanoparticle complex of claim 1, wherein the biological sample is any one or more selected from the group consisting of blood, saliva, bone marrow fluid, lymphatic fluid, urine, amniotic fluid, mucosal fluid, and peritoneal fluid.

11. A method for preparing the nanoparticle complex of claim 1, the method comprising the following steps:
a) preparing polydopamine-coated nanoparticles by adding and dispersing nanoparticles and an acid-base catalyst in a polydopamine solution; and
b) adding a receptor to the polydopamine-coated nanoparticles and reacting the resulting mixture to prepare a receptor-bound polydopamine-coated nanoparticle complex.

12. The method of claim 11, wherein the nanoparticles and polydopamine in Step a) are mixed at a weight ratio of 1:0.01 to 1:1.5.

13. The method of claim 11, wherein a monosaccharide is further added to the polydopamine solution in Step a) at a weight ratio of 1:0.5 to 1:1.5.

14. The method of claim 11, wherein the nanoparticles are iron oxide nanoparticles or polystyrene nanoparticles.

15. The method of claim 11, wherein in Step a), the nanoparticles and the acid-base catalyst are dispersed in the polydopamine solution for 4 hours to 36 hours.

16. The method of claim 11, wherein in Step b), the resulting mixture reacts for 1 hour to 5 hours.

17. A method for preparing the nanoparticle complex of claim 1, the method comprising the following steps:
a) preparing polymer material-coated nanoparticles by adding a polymer material to silica shell nanoparticles having a thiol group and dispersing the same; and
b) adding a receptor to the coated nanoparticles and reacting the resulting mixture to prepare a receptor-bound polymer material-coated nanoparticle complex bound,
wherein the polymer material is any one or more selected from the group consisting of polyethylene glycol, polyetherimide, polyvinyl alcohol, casein, dextran, and chitosan.

18. The method of claim 17, wherein in Step b), 1-ethyl-3-(3-dimethylaminopropyl)carbodiimide (EDC) and N-hydroxysuccinimide (NHS) are added along with the receptor.

19. The method of claim 17, wherein the nanoparticles and the polymer material in Step a) are mixed at a weight ratio of 1:1 to 1:5.

20. The method of claim 17, wherein in Step a), the polymer material is mixed and dispersed for 4 hours to 36 hours.

21. The method of claim 17, wherein in Step b), the resulting mixture reacts for 1 hour to 5 hours.

22. A kit for detecting or isolating a target material from a biological sample, comprising the nanoparticle complex of claim 1 as an active ingredient.

23. A kit for diagnosing an infectious disease, comprising the nanoparticle complex of claim 1 as an active ingredient.
